(19) United States
(12) Plant Patent Application Publication
(10) Pub. No.: US 2026/0020506 P1
(43) Pub. Date: Jan. 15, 2026

(54) Strawberry Plant Named 'Lady Isla'

(50) ;

(71) Applicant: S&A Soft Fruits Ltd., Marden (GB)

(72) Inventor: Dan Smith, Herefordshire (GB)

(21) Appl. No.: 18/770,604

(22) Filed: Jul. 11, 2024

(51) Int. Cl. A01H 6/74; A01H 5/08

(52) U.S. Cl. CPC A01H 6/7409; A01H 5/08
USPC PLT/208

(57) ABSTRACT

A new and distinct strawberry plant is provided that exhibits a semi-upright growth habit with medium to large, glossy fruit.

Botanical classification: Fragaria x ananassa (Duch.)

Varietal denomination: ‘Lady Isla’.

### BACKGROUND OF THE INVENTION

The present invention comprises a new and distinct variety of strawberry plant botanically classified as Fragaria x ananassa (Duch.) and known by the varietal name ‘Lady Isla’. The new variety was originally referred to as RD074-016-2012. The new variety is the result of a cross between strawberry seedling referred to as SA11 (female parent, unpatented) and strawberry seedling referred to as SA1 (male parent, unpatented). The resultant cross produced ‘Lady Isla’ in July of 2013 in Herefordshire, United Kingdom. The purpose of the breeding program was to develop novel low input, high yielding, everbearing, and day neutral strawberry varieties with excellent fruit quality and improved post-harvest longevity. Subsequently, the new variety was asexually reproduced via stolons in Herefordshire, United Kingdom in August of 2013. The new variety has been trial and field tested and has been found to retain its distinctive characteristics and remain true to type through successive asexual propagations. The new variety has not been evaluated under all possible environmental conditions. The phenotype may vary with variations in environment without a change in the genotype of the plant.

The new variety is similar to its female parent in having a high number of medium to large fruit per plant. However, ‘Lady Isla’ differs from its female parent in exhibiting fruit that are more conic in shape, whereas the fruit of the female parent are more cordate in shape. The new variety is similar to its male parent in conic fruit shape. However, the fruit of ‘Lady Isla’ are smaller and deeper red in color when ripe than the fruit of its male parent.

When compared to strawberry variety named ‘Lady Emma’ (U.S. Plant Pat. No. 32,466), ‘Lady Emma’ is remontant and very early to early with regard to the beginning of flowering, whereas ‘Lady Isla’ (also remontant) is slightly later. The fruit size of ‘Lady Emma’ is large to very large, whereas the fruit size of ‘Lady Isla’ is medium to large. The anthocyanin coloration of the stolon for ‘Lady Isla’ is medium to strong, whereas it is weak to medium for ‘Lady Emma’. The attitude of hairs on the pedicels of ‘Lady Isla’ are slightly outwards versus upwards for ‘Lady Emma’.

The following characteristics also distinguish the new variety from other strawberry varieties known to the breeder:
Semi-upright growth habit with medium to dense foliage;
Medium to large leaf size of a medium green color (adaxial surface) with strong leaf blistering present;
Medium to long petioles with horizontal attitude of pubescence;
Medium to large fruit with strong glossiness and medium red fruit color;
Early fruit ripening and strong day neutral behavior; and
Excellent fruit quality, pleasant aroma, sweet taste, and glossy berries.

### DESCRIPTION OF THE DRAWINGS

The accompanying photographic drawings illustrate the new variety at approximately 10 months of age, with the color being as nearly true as is possible with color illustrations of this type.

FIG. 1 is a photograph of multiple plants of the new variety;

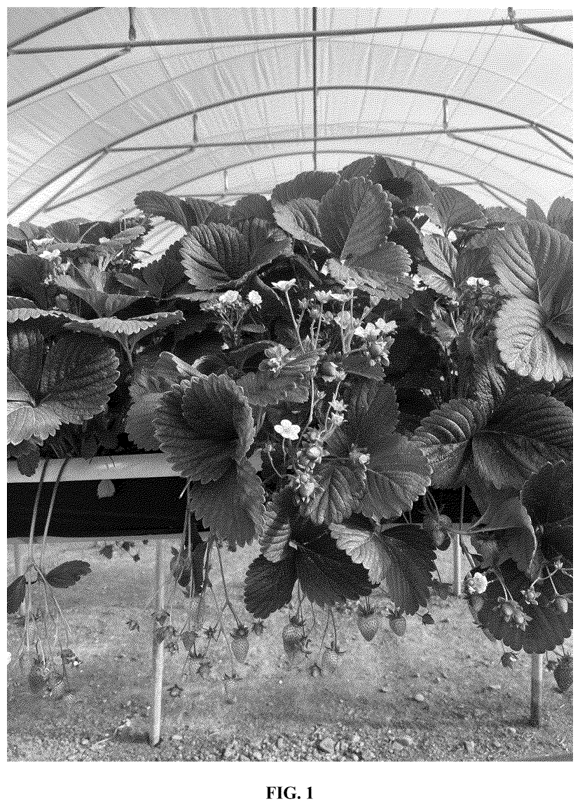

FIG. 2 is a photograph of whole fruits of the new variety;

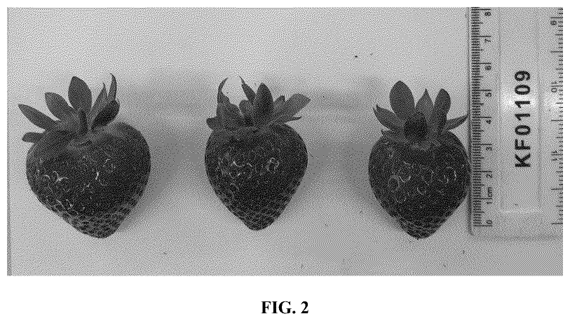

FIG. 3 is a photograph of halved fruits of the new variety; and

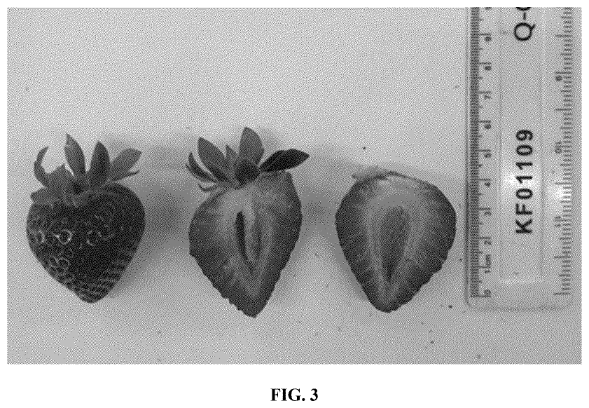

FIG. 4 is a photograph of flowers of the new variety.

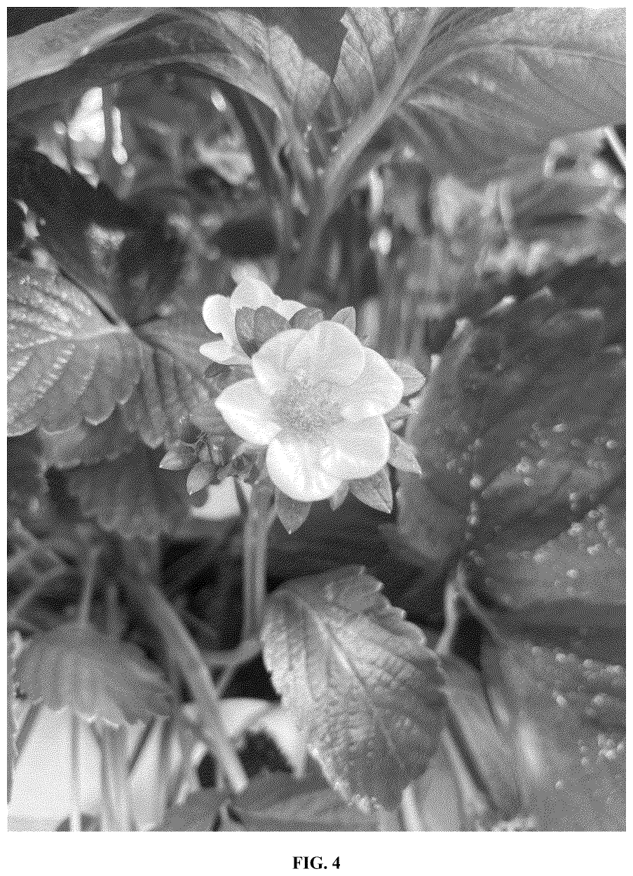

### DESCRIPTION OF THE PLANT

The following detailed description sets forth the characteristics of the new cultivar. The new variety was grown under polytunnel protection in 12.5 liter containers in Herefordshire, United Kingdom with an average day temperature of 18° C. and an average night temperature of 12° C. The new variety was approximately 9 months of age when described. Color references are primarily to the Sixth Edition (2015) of The R.H.S. Colour Chart of The Royal Horticultural Society of London and were identified under natural light.

### PLANT

Time to initiate roots: 1-4 days at an average temperature of 20° C.
Time to produce a rooted plant: 21-28 days at an average temperature of 20° C.
Time to produce a finished plant: 30-45 days at an average temperature of 20° C.
Rooting description: Vigorous, fibrous, and cream to white in color.
Plant form: Semi-upright with medium to dense growth.
Height (from soil to top of plant): Approximately 26 cm.
Plant diameter: 60 cm.
Vigor: Moderate to high.
Disease/pest resistance: Resistant to powdery mildew and crown rot.
Weather tolerance: Plants perform best in a protected environment, but are resistant to high temperature climates.
Foliage:
   Arrangement.—Basal rosette; upwardly forming.
   Average number of leaves per stem.—1.
   Whole leaf length.—7-11 cm.
   Whole leaf width.—9-15 cm.
   Petiole.—Length: 13-24 cm. Diameter: 2-5 mm. Color: RHS 145B. Texture: Pubescence outwardly facing.
   Stipule.—Average number per leaf: 2.Length: 10-15 mm. Width:2-10 mm. Color: RHS 145C.
   Color.—Young leaflets: Upper surface: RHS 143A. Lower surface: RHS 139C. Mature leaflets: Upper surface: RHS 137A. Lower surface: RHS 147C.
   Lateral leaflets.—Length: 5-10 cm. Width: 4-8 cm. Shape of leaf (generally): Typically orbicular, basally asymmetrical, cross-sectionally concave at the base changing to convex at the apex. Shape of apex: Truncate. Shape of base: Oblique/obtuse. Texture (both surfaces): Lightly pubescent. Aspect: Upright. Margin type: Crenate.
   Terminal leaflets.—Length: 6-10 cm. Width: 5-8 cm. Shape of leaf (generally): Cross-sectionally concave. Shape of apex: Truncate. Shape of base: Acute. Texture (both surfaces): Lightly pubescent. Aspect: Upright. Margin type: Crenate.
   Veins.—Venation pattern: Pinnate. Color: Upper surface: RHS 144D. Lower surface: Between RHS 145C and 145D.
Fruit:
   Harvest season.—From April-December in Herefordshire, United Kingdom.
   Number of fruit per fruiting lateral.—Typically 5-10.
   Surface color.—Immature: RHS 150D. Maturing: RHS 40D. Fully mature: RHS 44A. Evenness of color: Slightly uneven.
   Taste.—Excellent.
   Aroma.—Sweet.
   Length.—30-46 mm.
   Width.—26-39 mm.
   Length/width ratio.—Longer than wide.
   Overall shape.—Conical.
   Glossiness.—Strong.
   Weight.—Average of 15 grams across a growing season.
   Achene position.—At surface level.
   Achene color.—RHS 162B.
   Average number of achenes per berry.—240.
   Flesh firmness.—Medium to firm.
   Fruiting truss length.—25-40 cm.
   Fruiting truss diameter.—2-6 mm.
   Fruiting truss color.—RHS N144D.
Reproductive organs:
   Stamens.—Number per flower: 19-26.Length: 3-6 mm. Width: 1-2 mm.
   Anthers.—Apex shape: Rounded. Base shape: Cordate. Length: 1-2 mm. Width: 1-2 mm. Color: Edge: RHS 15B. Center: RHS 7A.
   Stigma.—Texture: Lightly papillose. Shape: Rounded to acute. Color: RHS 153D.
   Style.—Length: Approximately 1.2 mm. Color: RHS 4A.
   Ovary.—Texture: Pubescence present. Length: Approximately 0.5 mm. Color: With seed: RHS 150C. Without seed: RHS 144B.
   Stolon.—Length: Mother plant to first stolon-plant: 57 cm. Mother plant to second stolon-plant: 100 cm. Diameter: Approximately 20 mm. Color: RHS 145C.
Flowers:
   Natural flowering season.—From March-December in Herefordshire, United Kingdom.
   Number of flowers per plant.—120-180.
   Fragrance.—None present.
   Longevity.—4-7 days.
   Flower description.—Rotate flowers arranged singly at lateral apices that are cupped slightly upwards with moderate overlap and are alternatively concave/convex.
   Flower height.—Primary: Average 10.6 mm, range 6-15 mm. Secondary: Average 7.9 mm, range 5-10 mm.
   Flower diameter.—Primary: Average 32.1 mm, range 23-40 mm. Secondary: Average 24.4 mm, range 20-29 mm.
   Petals.—Number per flower: 5. Shape: Globose to obovate. Length: Average 12.2 mm, range 10-15 mm. Width: Average 13 mm, range 11-16 mm. Apex: Rounded. Base: Acute point. Margin: Entire. Texture (both surfaces): Smooth, with no pubescence present. Color: Mature petal: Upper surface: RHS 155B. Lower surface: RHS 155C. Young petal: Upper surface: RHS 155C. Lower surface: RHS 155C.
   Sepals.—Number per flower: Average 10, range 10-12. Length: Average 11.1 mm, range 7-19 mm. Width: Average 5.7 mm, range 3-12 mm. Shape: Lanceolate with typically a single apex; can be multi-pointed on primary flowers. Apex: Acute. Margin: Typically entire; can be serrated on primary flowers. Texture: Pubescence present on both upper and lower surfaces. Color: Mature sepal: Upper surface: RHS 138A. Lower surface: RHS 138C. Young sepal: Upper surface: RHS 141B. Lower surface: RHS 143C.
   Buds.—Length: Average 16.6 mm, range 12-21 mm. Diameter: Average 8.4 mm, range 6-12 mm. Shape: Ovoid. Color: RHS N144A.

I claim:

1. A new and distinct variety of strawberry plant named ‘Lady Isla’, as is hercin illustrated and described.
